(19) United States
(12) Patent Application Publication
(10) Pub. No.: US 2026/0021246 A1
(43) Pub. Date: Jan. 22, 2026

(54) CLAMPING DEVICE AND ELECTRONIC DEVICE INCLUDING THE SAME

(71) Applicant: InnoLux Corporation, Miao-Li County (TW)

(72) Inventors: Huang-Ren YAO, Miao-Li County (TW); Ming-Han LIN, Miao-Li County (TW); Shao-Yu CHIU, Miao-Li County (TW)

(21) Appl. No.: 19/244,556

(22) Filed: Jun. 20, 2025

Related U.S. Application Data
(60) Provisional application No. 63/673,378, filed on Jul. 19, 2024.

(30) Foreign Application Priority Data
Mar. 20, 2025 (CN) 202510332615.2

(51) Int. Cl. A61M 5/14; A61M 5/145; A61M 5/172

(52) U.S. Cl. CPC A61M 5/1413; A61M 5/1456; A61M 5/172

(57) ABSTRACT

A clamping device is used to clamp a syringe and includes: a first fixing member and a second fixing member; a first pressing member and a second pressing member; and a first connecting member and a second connecting member, wherein two ends of the first connecting member are pivotally connected to the first fixing member and the first pressing member, respectively, and two ends of the second connecting member are pivotally connected to the second fixing member and the second pressing member, respectively, wherein the syringe includes a barrel and a handle and, when the first fixing member and the second fixing member approach to each other and contact the barrel of the syringe, the first pressing member and the second pressing member contact the handle of the syringe.

### CROSS-REFERENCE TO RELATED APPLICATIONS

This application claims the benefit of filing date of U.S. Provisional Application Ser. No. 63/673,378 filed on Jul. 19, 2024 under 35 USC § 119 (e) (1), and also claims the benefit of the Chinese Patent Application Serial Number 202510332615.2, filed on Mar. 20, 2025, the subject matters of which are incorporated herein by reference.

### BACKGROUND

### Field of the Disclosure

The present disclosure relates to a clamping device and an electronic device including the same and, more particularly, to a clamping device for clamping a syringe and an electronic device including the same.

### Description of Related Art

With the advancement of science and technology, the fields of automated operations are increasing and the scope of coverage is becoming wider and wider. Drug dispensing equipment may be used to administer drugs in hospitals. When drugs are toxic, drug dispensing equipment may reduce the risk of medical staff coming into contact with highly toxic drugs, save labor costs and/or reduce human errors, thereby gradually attracting attention from the medical community.

However, since syringes are provided with various sizes, when used in conjunction with automation, syringe holders of different specifications must be replaced to match syringes of different sizes, resulting in a cumbersome operation process. Additionally, replacing the syringe holder may be time consuming and/or costly.

Therefore, there is an urgent need to provide a device to alleviate and/or obviate the aforementioned defects.

### SUMMARY

The present disclosure provides a clamping device for clamping a syringe, which includes: a first fixing member and a second fixing member; a first pressing member and a second pressing member; and a first connecting member and a second connecting member, wherein two ends of the first connecting member are pivotally connected to the first fixing member and the first pressing member, respectively, and two ends of the second connecting member are pivotally connected to the second fixing member and the second pressing member, respectively, wherein the syringe includes a barrel and a handle and, when the first fixing member and the second fixing member approach to each other and contact the barrel of the syringe, the first pressing member and the second pressing member contact the handle of the syringe.

The present disclosure further provides an electronic device, which includes: a clamping device for clamping a syringe, including: a first fixing member and a second fixing member; a first pressing member and a second pressing member; and a first connecting member and a second connecting member, wherein two ends of the first connecting member are pivotally connected to the first fixing member and the first pressing member, respectively, and two ends of the second connecting member are pivotally connected to the second fixing member and the second pressing member, respectively; and a push-pull device adjacent to the clamping device, including: a first push rod clamp and a second push rod clamp; and a push rod clamp driver for driving the first push rod clamp and the second push rod clamp, wherein the syringe includes a barrel, a handle and a push rod and, when the first fixing member and the second fixing member approach to each other and contact the barrel of the syringe, the first pressing member and the second pressing member contact the handle of the syringe, and the first push rod clamp and the second push rod clamp approach each other and contact the push rod of the syringe.

Other novel features of the disclosure will become more apparent from the following detailed description when taken in conjunction with the accompanying drawings.

### BRIEF DESCRIPTION OF DRAWINGS

FIG. 1A is a block diagram of a drug dispensing process according to an embodiment of the present disclosure;

FIG. 1B is another block diagram of the drug dispensing process according to an embodiment of the present disclosure,

FIG. 2A is a three-dimensional schematic diagram of a clamping device according to an embodiment of the present disclosure;

FIG. 2B is an exploded view of the clamping device of FIG. 2A;

FIG. 2C is a partial cross-sectional view of the clamping device taken along line A-A′ of FIG. 2A;

FIG. 3 is a schematic diagram of a syringe according to an embodiment of the present disclosure;

FIG. 4 is a block diagram illustrating the operation flow of the clamping device according to an embodiment of the present disclosure;

FIG. 5 is a three-dimensional schematic diagram of an electronic device according to an embodiment of the present disclosure;

FIG. 6A is a three-dimensional schematic diagram of a push-pull device according to an embodiment of the present disclosure;

FIG. 6B is a schematic side view of the push-pull device of FIG. 6A; and

FIG. 6C is an exploded view of the push-pull device of FIG. 6A.

### DETAILED DESCRIPTION OF EMBODIMENT

The electronic device according to the embodiment of the present disclosure is described in detail below. It should be understood that the following description provides many different embodiments for implementing different aspects of some embodiments of the present disclosure. The specific components and arrangements described below are only for the purpose of simply and clearly describing some embodiments of the present disclosure. Of course, these are only examples and are not limitations of the present disclosure. In addition, similar and/or corresponding reference numerals may be used in different embodiments to identify similar and/or corresponding components in order to clearly describe the present disclosure. However, the use of these similar and/or corresponding reference numerals is only for simply and clearly describing some embodiments of the present disclosure, and does not represent any relationship between the different embodiments and/or structures discussed.

The embodiments of the present disclosure may be understood together with the drawings, and the drawings of the present disclosure are also regarded as part of the disclosure description. It should be understood that the drawings of the present disclosure are not in scale and, in fact, the dimensions of elements may be arbitrarily enlarged or reduced in order to clearly illustrate features of the present disclosure. In addition, directional terms mentioned in the present disclosure, such as “up”, “down”, “front”, “rear”, “left”, “right”, etc., only refer to the directions of the drawings. Accordingly, the directional term used is illustrative, not limiting, of the present disclosure. In the drawings, various figures illustrate the general characteristics of methods, structures and/or materials used in particular embodiments. However, these drawings should not be construed to define or limit the scope or nature encompassed by these embodiments. For example, the relative sizes, thicknesses and positions of various layers, regions and/or structures may be reduced or enlarged for clarity.

One structure (or layer, component, substrate) described in the present disclosure is disposed on/above another structure (or layer, component, substrate), which can mean that the two structures are adjacent and directly connected, or can refer to two structures that are adjacent rather than directly connected. Indirect connection means that there is at least one intermediate structure (or intermediate layer, intermediate component, intermediate substrate, intermediate space) between the two structures, the lower surface of one structure is adjacent to or directly connected to the upper surface of the intermediate structure, and the upper surface of the other structure is adjacent to or directly connected to the lower surface of the intermediate structure. The intermediate structure may be a single-layer or multi-layer physical structure or a non-physical structure, which is not limited. In the present disclosure, when a certain structure is arranged “on” other structures, it may mean that a certain structure is “directly” on other structures, or it means that a certain structure is “indirectly” on other structures; that is, at least one structure is sandwiched, in between a certain structure and other structures. In the present disclosure, the term “correspondingly disposed” or “disposed corresponding to” refers to, for example, that the components substantially overlap each other, but the present disclosure is not limited thereto.

In addition, it should be understood that, in the specification and claims, unless otherwise specified, ordinal numbers, such as “first” and “second”, used herein are intended to distinguish elements rather than disclose explicitly or implicitly that names of the elements bear the wording of the ordinal numbers. The ordinal numbers do not imply what order an element and another element are in terms of space, time or steps of a manufacturing method. Thus, what is referred to as a “first element” in the specification may be referred to as a “second element” in the claims

In some embodiments of the present disclosure, terms such as “connection” and “interconnection” about joining and connecting, unless otherwise specified, may mean that two structures are in direct contact, or may also mean that two structures are not in direct contact, where other structures are placed between the two structures. Moreover, the terms about joining and connecting may include the situation that both structures are movable, or both structures are fixed. In addition, the term “coupled” includes any direct and indirect means of electrical connection. The term “pivoted” or “pivotally connected” refers to two elements that can rotate relative to each other via an axis.

In the description, the terms “almost”, “about”, “approximately” or “substantially” usually means within 10%, 5%, 3%, 2%, 1% or 0.5% of a given value or range. Unless otherwise defined, the term “range between the first value and the second value” indicates that the range includes the first value, the second value, and other values in between. Moreover, any two values or directions used for comparison may have certain errors. If the first value is equal to the second value, it implies that there may be an error of about 10% between the first value and the second value; if the first direction is perpendicular or “approximately” perpendicular to the second direction, the angle between the first direction and the second direction may be between 80 degrees and 100 degrees; if the first direction is parallel or “substantially” parallel to the second direction, the angle between the first direction and the second direction may be between 0 degrees and 10 degrees. In the present disclosure, the expressions “the given range is from the first value to the second value” and “the given range falls within the range from the first value to the second value” indicate that the given range includes the first value, the second value, and other values in between.

In the entire specification and appended claims of the present disclosure, certain words are used to refer to specific components. Those skilled in the art should understand that electronic device manufacturers may refer to the same components by different names. The present disclosure does not intend to distinguish those components with the same function but different names. In the following description and claims, words such as “comprising”, “including”, and “having” are open type words, so they should be interpreted as meaning “including but not limited to”. Therefore, when the terms “comprising”, “including” and/or “having” are used in the description of the present disclosure, they specify the existence of corresponding features, regions, steps, operations and/or components, but do not exclude the existence of one or more corresponding features, regions, steps, operations and/or components.

In addition, the clamping device of the present disclosure may be applied to the electronic device itself or the application of the electronic device. The electronic device may include a display device, an automation device, a clamping device, a mobile platform picking device, a computing device, a mechanical device, a drug dispensing device, an exposure device, a printing device, a three-dimensional printing device, a vehicle device, an imaging device, an assembly device, a backlight device, an antenna device, a tiled device, a touch electronic device, a curved electronic device or a free shape electronic device, but not limited thereto. The display device may include, for example, liquid crystal, light emitting diode, fluorescence, phosphor, other suitable display media, or a combination thereof, but not limited thereto. The display device may be a non-self-luminous display device or a self-luminous display device. The antenna device may be a liquid crystal type antenna device or a non-liquid crystal type antenna device, and the sensing device may be a sensing device that senses capacitance, light, heat energy, or ultrasound, but not limited thereto. The tiled device may include, for example, a display tiled device or an antenna tiled device, but not limited thereto. It should be noted that the electronic device may be any arrangement or combination of the aforementioned, but not limited thereto. In addition, the electronic device may be a bendable or flexible electronic device. It should be noted that the electronic device may be any arrangement or combination of the aforementioned, but not limited thereto. In addition, the shape of the electronic device may be rectangular, circular, polygonal, a shape with curved edges, or other suitable shapes. The electronic device may have a peripheral system such as a driving system, a control system, a light source system, a shelf system, etc. to support a display device, an antenna device, or a tiled device. The electronic device may include, for example, electronic components, liquid crystal, light emitting diode, quantum dot (QD), fluorescence, phosphor, other suitable display media, or a combination thereof, but not limited thereto. The electronic components may include passive components and active components, such as capacitors, resistors, inductors, diodes, transistors, etc. The diode may include a light emitting diode or a photodiode. The light emitting diode may include, for example, an organic light emitting diode (OLED), a sub-millimeter light emitting diode (mini LED), a micro LED or a quantum dot light emitting diode (which may include QLED, QDLED), a light emitting diode for a flexible display, or other suitable materials, or a combination thereof, but not limited thereto.

It should be understood that, without departing from the spirit of the present disclosure, in the following embodiments, the features in different embodiments may be replaced, reorganized or mixed to accomplish other embodiments. The features among various embodiments may be mixed and matched arbitrarily as long as they do not violate the spirit of the invention or conflict with each other.

Unless otherwise defined, all terms (including technical and scientific terms) used here have the same meanings as commonly understood by those skilled in the art of the present disclosure. It is understandable that these terms, such as those defined in commonly used dictionaries, should be interpreted as having a meaning consistent with the relevant technology and the background or context of the present disclosure, rather than in an idealized or excessively formal interpretation, unless specifically defined in the embodiment of the present disclosure.

It should be noted that the following embodiments may be implemented by replacing, reorganizing, or mixing the features of several different embodiments without departing from the spirit of the present disclosure to implement other embodiments. The features of the various embodiments may be mixed and matched as desired as long as they do not violate the spirit of the present disclosure or conflict with each other. It should be noted that the technical solutions provided in the following different embodiments may be replaced, combined or mixed with each other to form another embodiment without violating the spirit of the present disclosure.

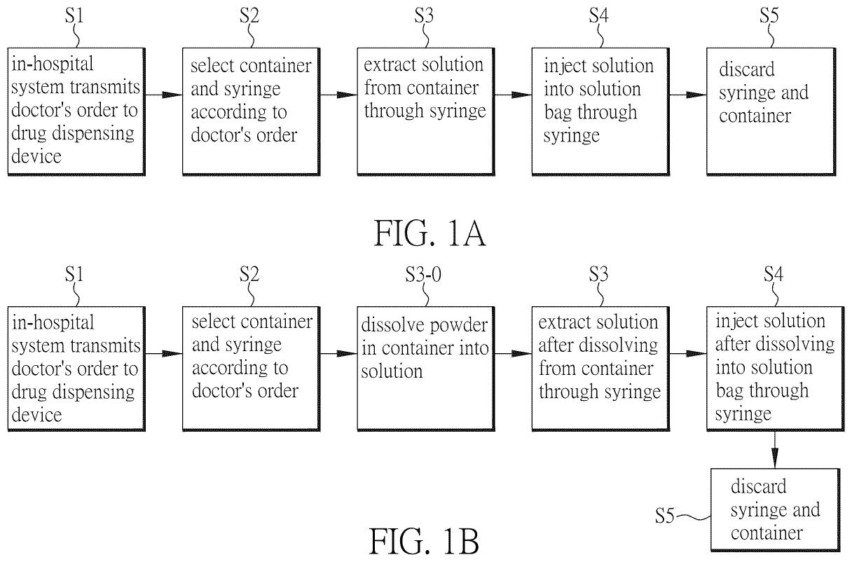

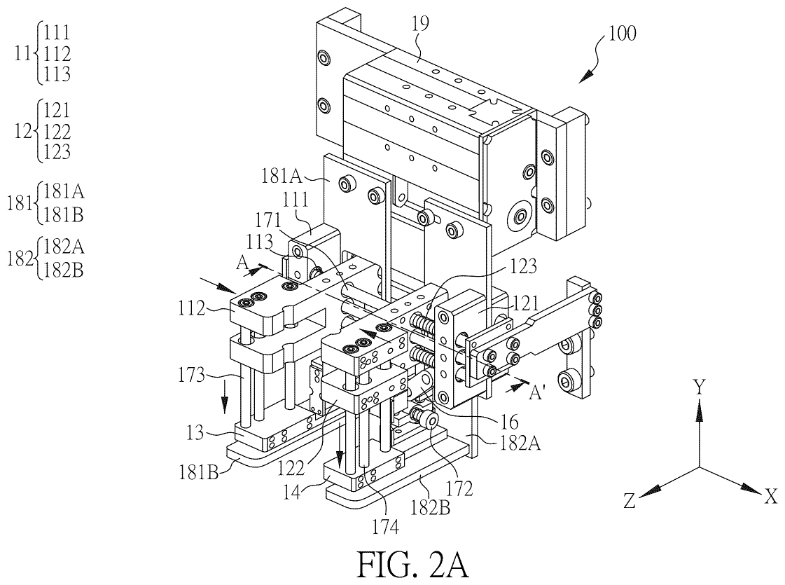

FIG. 1A is a block diagram of a drug dispensing process according to an embodiment of the present disclosure.

In one embodiment of the present disclosure, as shown in FIG. 1A, the drug dispensing process may include: step S1, in which the in-hospital system transmits a doctor's order (for example, a prescription) to a drug dispensing device. Next, step S2 is executed, in which the operator of the drug dispensing device or the drug dispensing device itself (if the drug dispensing device has an automated function) selects an appropriate container and syringe according to the doctor's order. The container may contain a solution of a medication, a nutritional supplement, and/or other ingredients. The drugs include, for example, chemotherapy drugs, analgesics, other drugs suitable for intravenous injection, or a combination thereof, but the present disclosure is not limited thereto. The nutritional supplements include, for example, sugar, vitamins, potassium ions, other suitable nutritional additives, or a combination thereof, but the present disclosure is not limited thereto.

Afterwards, step S3 is executed, in which the drug dispensing device extracts the solution from the container through the syringe. Next, step S4 is performed, in which the drug dispensing device injects the solution into the solution bag through the syringe. Finally, step S5 is executed, in which the drug dispensing device discards the syringe and the container to complete the drug dispensing. When executing step S3 and step S4, the clamping device of the present disclosure or an electronic device containing the same may be applied to realize an automated drug dispensing process, thereby achieving the advantages of reducing the risk of medical staff in contact with drugs, alleviating the burden on medical staff and/or reducing human errors.

FIG. 1B is another block diagram of the drug dispensing process according to an embodiment of the present disclosure, wherein the drug dispensing process of FIG. 1B is similar to that of FIG. 1A, except for the following differences.

In one embodiment of the present disclosure, as shown in FIG. 1B, when the ingredients in the container include solid powder, the drug dispensing process may further include: before step S3, step S3-0, in which the powder in the container is dissolved into a solution. Afterwards, step S3 to step S5 are sequentially executed to complete the drug dispensing. When executing step S3 and step S4, the clamping device of the present disclosure or an electronic device including the same may be applied to realize an automated drug dispensing process, thereby achieving the advantages of reducing the risk of medical staff in contact with drugs, alleviating the burden on medical staff and/or reducing human errors.

The clamping device 100 of the present disclosure and the electronic device including the same will be described in detail below.

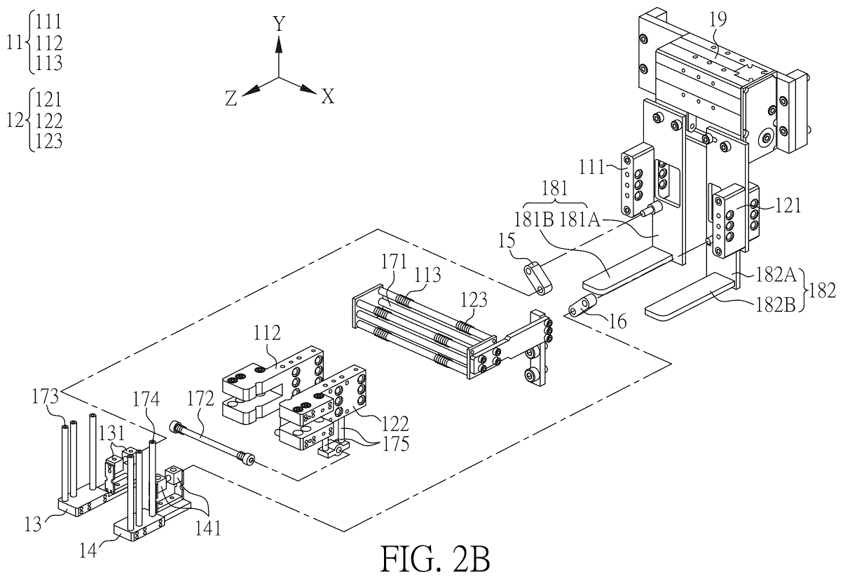

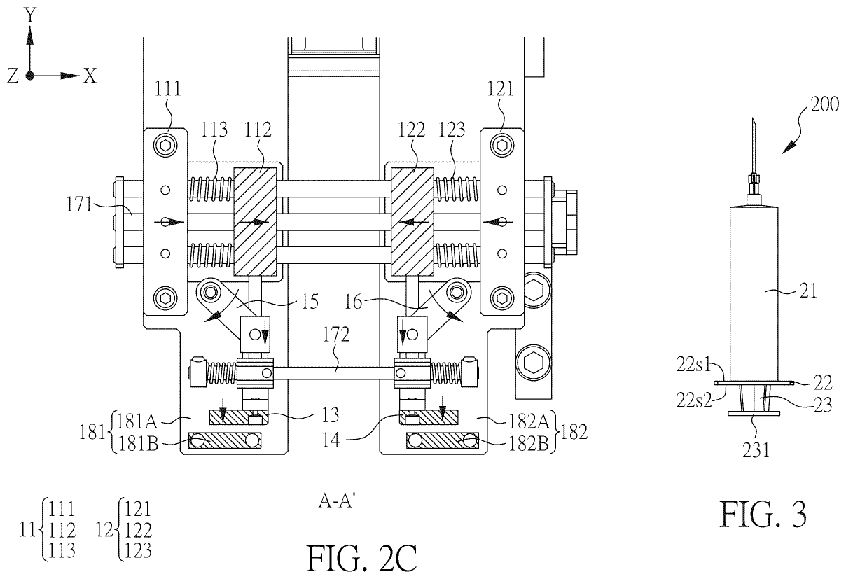

FIG. 2A is a three-dimensional schematic diagram of a clamping device according to an embodiment of the present disclosure. FIG. 2B is an exploded view of the clamping device of FIG. 2A. FIG. 2C is a partial cross-sectional view of the clamping device taken along line A-A′ of FIG. 2A. FIG. 3 is a schematic diagram of a syringe according to an embodiment of the present disclosure.

In one embodiment of the present disclosure, as shown in FIG. 2A to FIG. 2C, the clamping device may include: a first fixing member 11 and a second fixing member 12; a first pressing member 13 and a second pressing member 14; and a first connecting member 15 and a second connecting member 16, wherein the two ends of the first connecting member 15 are pivotally connected to the first fixing member 11 and the first pressing member 13, respectively, and the two ends of the second connecting member 16 are pivotally connected to the second fixing member 12 and the second pressing member 14, respectively. In more detail, the dotted line connections in FIG. 2B respectively indicate the pivotal connecting positions of the first connecting member 15 and the first fixing member 11 and the first pressing member 13, and the pivotal connecting positions of the second connecting member 16 and the second fixing member 12 and the second pressing member 14.

In the present disclosure, the first fixing member 11 includes a first base body 111, a first contact member 112 and a first elastic element 113, wherein the first elastic element 113 is arranged between the first base body 111 and the first contact member 112. The second fixing member 12 includes a second base body 121, a second contact member 122 and a second elastic element 123, wherein the second elastic element 123 is arranged between the second base body 121 and the second contact member 122. The first base body 111, the second base body 121, the first contact member 112 and the second contact member 122 are respectively connected in a sliding manner to a main guide rod 171 extending laterally (such as in the X direction), wherein the first elastic element 113 and the second elastic element 123 are respectively arranged on the main guide rod 171, and the first elastic element 113 is disposed between the first base body 111 and the first contact member 112, and the second elastic element 123 is disposed between the second base body 121 and the second contact member 122. The laterally extending main guide rod 171 allows the first base body 111, the second base body 121, the first contact member 112 and the second contact member 122 to move laterally; that is, the first base body 111, the second base body 121, the first contact member 112 and the second contact member 122 may move along the first direction (for example, the +X direction) and/or along a direction opposite to the first direction (for example, the −X direction). In one embodiment of the present disclosure, as shown in FIG. 2B, the first contact member 112 and the second contact member 122 may be respectively connected in a sliding manner to an auxiliary guide rod 172 extending in a transverse direction (for example, in the X direction), and the auxiliary guide rod 172 allows the first contact member 112 and the second contact member 122 to move in a transverse direction; that is, the first contact member 112 and the second contact member 122 may move in a first direction (for example, in the +X direction) and/or in a direction opposite to the first direction (for example, in the −X direction).

In the present disclosure, as shown in FIG. 2A to FIG. 2C, the clamping device 100 may include a first back plate 181 and a second back plate 182, wherein the first base body 111 is fixed on the first back plate 181, and the second base body 121 is fixed on the second back plate 182. In more detail, the first back plate 181 may include a first portion 181A and a second portion 181B connected to the first portion 181A, wherein the first portion 181A extends along a second direction (for example, Y direction) and the second portion 181B extends along a third direction (for example, Z direction). The second back plate 182 includes a third portion 182A and a fourth portion 182B connected to the third portion 182A, wherein the third portion 182A extends along the second direction (for example, the Y direction), and the fourth portion 182B extends along the third direction (for example, the Z direction). The first base body 111 is fixed on the first portion 181A of the first back plate 181, and the second base body 121 is fixed on the third portion 182A of the second back plate 182. In one embodiment of the present disclosure, the first direction (for example, X direction) is different from the second direction (for example, Y direction) and the third direction (for example, Z direction). For example, the first direction (for example, X direction) is perpendicular to the second direction (for example, Y direction), and the first direction (for example, X direction) is perpendicular to the third direction (for example, Z direction). In one embodiment of the present disclosure, the second direction (for example, Y direction) is different from the third direction (for example, Z direction). For example, the second direction (for example, Y direction) is perpendicular to the third direction (for example, Z direction).

In one embodiment of the present disclosure, as shown in FIG. 2B, a first guide rod 173 extending longitudinally (for example, in the Y direction) is provided on the first pressing member 13, and a second guide rod 174 extending longitudinally is provided on the second pressing member 14. The first pressing member 13 is connected in a sliding manner to the first contact member 112 through the first guide rod 173, and the second pressing member 14 is connected in a sliding manner to the second contact member 122 through the second guide rod 174. In addition, a third guide rod (not shown) extending longitudinally may be provided on the first contact member 112, and a fourth guide rod 175 extending longitudinally may be provided on the second contact member 122. A first sliding member 131 is provided on the first pressing member 13, and a second sliding member 141 is provided on the second pressing member 14. The first pressing member 13 may be connected in a sliding manner to the first contact member 112 through the first sliding member 131 and the third guide rod (not shown), and the second pressing member 14 may be connected in a sliding manner to the second contact member 122 through the second sliding member 141 and the fourth guide rod 175. Therefore, the first guide rod 173, the second guide rod 174, the third guide rod and the fourth guide rod 175 respectively allow the first pressing member 13 and the second pressing member 14 to move in the longitudinal direction; that is, the first pressing member 13 and the second pressing member 14 may move along the second direction (for example, −Y direction) and/or in a direction opposite to the second direction (for example, +Y direction).

In the present disclosure, the main guide rod 171 is exemplified as a group including six guide rods, but the present disclosure is not limited thereto. In other aspects, the main guide rod 171 may include, for example, one, two, three or other suitable numbers of guide rods. In the present disclosure, the auxiliary guide rod 172 is illustrated as including one guide rod, but the present disclosure is not limited thereto. In other aspects, the auxiliary guide rod 172 may include, for example, two, three, or other suitable numbers of guide rods. In the present disclosure, the first guide rod 173 is exemplified as a group including three guide rods, but the present disclosure is not limited thereto. In other aspects, the first guide rod 173 may include, for example, one, two, or other suitable numbers of guide rods. Similarly, the second guide rod 174 is exemplified as a group including three guide rods, but the present disclosure is not limited thereto. In other aspects, the second guide rod 174 may include, for example, one, two, or other suitable numbers of guide rods. In the present disclosure, the fourth guide rod 175 is exemplified as a group including two guide rods, but the present disclosure is not limited thereto. In other aspects, the fourth guide rod 175 may include, for example, one, three or other suitable numbers of guide rods. In addition, although not shown in the figures, the third guide rod may have a design similar to that of the fourth guide rod 175, and thus a detailed description is deemed unnecessary.

In one embodiment of the present disclosure, as shown in FIG. 3, a syringe 200 may include a barrel 21, a handle 22 and a push rod 23, wherein the barrel 21 has a space for accommodating a sample (such as a solution), and the space has an opening into which the push rod 23 may be inserted. The size of the space may be changed by changing the position of the push rod 23 in the space, so as to control the injection or extraction of the sample, and the handle 22 is disposed on one side of the barrel 21. Specifically, the handle 22 is disposed adjacent to the opening of the space. The push rod 23 has a push rod handle 231 at its end.

The clamping device 100 of the present disclosure may be used to clamp the syringe 200. When the first fixing member 11 and the second fixing member 12 approach to each other and contact the barrel 21 of the syringe 200, the first pressing member 13 and the second pressing member 14 contact the handle 22 of the syringe 200. In more detail, the first fixing member 11 contacts the barrel 21 of the syringe 200 through the first contact member 112, and the second fixing member 12 contacts the barrel 21 of the syringe 200 through the second contact member 122, thereby clamping and fixing the barrel 21 of the syringe 200; the first pressing member 13 and the second pressing member 14 contact one side 22s1 of the handle 22 of the syringe 200, and the second portion 181B of the first back plate 181 and the fourth portion 182B of the second back plate 182 contact the other side 22s2 of the handle 22 of the syringe 200, thereby clamping and fixing the handle 22 of the syringe 200, thereby achieving the purpose of clamping the syringe 200. By adjusting the distance between the first fixing member 11 and the second fixing member 12, the clamping device 100 of the present disclosure may be suitable for syringes 200 of various sizes. Therefore, the cost of purchasing clamping devices for syringes of different sizes may be reduced and/or the time of replacing clamping devices for syringes of different sizes during operation may be reduced, thereby achieving the effect of saving costs and/or simplifying operating steps. In the present disclosure, “a side 22sl of the handle 22” refers to, for example, a side of the handle 22 close to the barrel body 21, or a side of the handle 22 away from the push rod handle 231 of the push rod 23, and “the other side 22s2 of the handle 22” refers, for example, to the side of the handle 22 away from the barrel body 21, or the side of the handle 22 close to the push rod handle 231 of the push rod 23.

In one embodiment of the present disclosure, as shown in FIG. 2A and FIG. 2B, the clamping device 100 may further include a clamping driver 19 for driving the first fixing member 11 and the second fixing member 12 to move the first fixing member 11 and the second fixing member 12 closer to or farther from each other. In more detail, as shown in FIG. 2C and FIG. 3, when the clamping driver 19 (as shown in FIG. 2A) drives the first base body 111 of the first fixing member 11 and the second base body 121 of the second fixing member 12, the first base body 111 will push the first contact member 112 so that both of them approach to the second fixing member 12 in the first direction (for example, the +X direction), and the second base body 121 will also simultaneously push the second contact member 122 so that both of them approach to the first fixing member 11 in a direction opposite to the first direction (for example, the −X direction), thereby allowing the first fixing member 11 and the second fixing member 12 to approach to each other along the first direction (that is, the X direction) so as to clamp the syringe 200. After the first contact member 112 and the second contact member 122 each contact the barrel 21 of the syringe 200, the clamping driver 19 will continue to drive the first base 111 and the second base 121 to allow the first base body 111 and the second base body 121 to continue to approach each other along the first direction (that is, the X direction). Since the first contact member 112 and the second contact member 122 are restricted by the barrel 21 of the syringe 200 and thus stop moving, the first base body 111 and the second base body 121 continue to approach each other, which will cause the first elastic element 113 and the second elastic element 123 to be compressed by stress, respectively, and the first connecting member 15 and the second connecting member 16 are respectively pushed by the first base body 111 and the second base body 121 to rotate. The first connecting member 15 and the second connecting member 16 convert the thrust direction by rotating. Specifically, the first pressing member 13 and the second pressing member 14 may contact the handle 22 of the syringe 200 in the second direction (for example, −Y direction). More specifically, the first base body 111 pushes the first connecting member 15 in the first direction (for example, +X direction), and the first connecting member 15 converts the thrust direction by rotating to move the first pressing member 13 in a second direction (for example, −Y direction). The second base body 121 pushes the second connecting member 16 in a direction opposite to the first direction (for example, −X direction), and the second connecting member 16 converts the thrust direction by rotating to move the second pressing member 14 in the second direction (for example, −Y direction), so that the first pressing member 13 and the second pressing member 14 contact the handle 22 of the syringe 200. In the present disclosure, the rotation direction of the first connecting member 15 is different from the rotation direction of the second connecting member 16; for example, the first connecting member 15 rotates clockwise, and the second connecting member 16 rotates counterclockwise, but the present disclosure is not limited thereto. In the present disclosure, a single driving source (for example, a clamping driver 19) is used to drive the first fixing member 11 and the second fixing member 12 to move in a first direction (for example, +X direction) and/or in a direction opposite to the first direction (for example, −X direction), and the thrust direction is converted through the first connecting member 15 and the second connecting member 16 to move the first pressing member 13 and the second pressing member 14 in a second direction (for example, −Y direction). As a result, the purpose of clamping and fixing the syringe 200 can be achieved. Without replacing the first fixing member 11 or the second fixing member 12, it is able to clamp and fix syringes 200 of different sizes or dimensions, thereby achieving the effects of saving costs (for example, reducing the driving source) and/or simplifying the operating steps.

In the present disclosure, before the clamping device 100 clamps the syringe 200, the first elastic element 113 and the second elastic element 123 will be pushed by the first base body 111 and the second base body 121 respectively to contact the first contact member 112 and the second contact member 122, thereby pushing the first contact member 112 and the second contact member 122. When the first contact member 112 and the second contact member 122 are respectively in contact with the barrel 21 of the syringe 200, the clamping driver 19 continues to drive the first base body 111 and the second base body 121, and the first elastic element 113 and the second elastic element 123 will be respectively compressed by stress. In other words, the lengths of the first elastic element 113 and the second elastic element 123 will be shortened at this moment. Therefore, after the clamping device 100 clamps the syringe 200, the sizes of the first elastic element 113 and the second elastic element 123 will be smaller than the initial sizes, wherein the “size of the elastic element” refers to, for example, the length of the elastic element in a first direction (for example, +X direction), but it is not limited thereto.

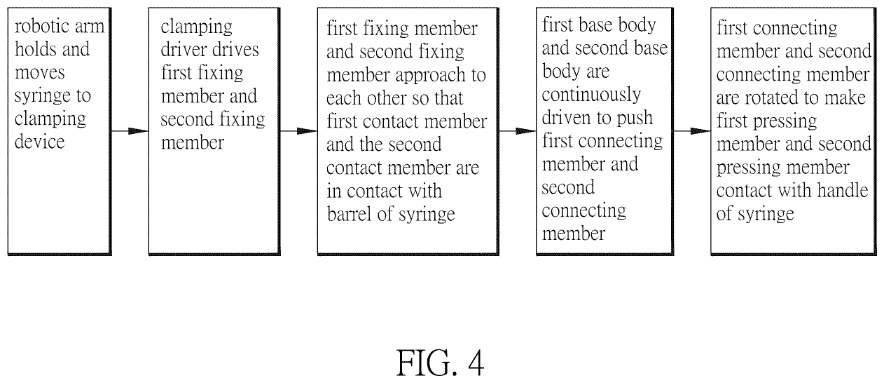

FIG. 4 is a block diagram illustrating the operation flow of the clamping device according to an embodiment of the present disclosure. The clamping device 100 may be, for example, as shown in FIG. 2A to FIG. 2C, and the syringe 200 may be, for example, as shown in FIG. 3, which will not be described in detail below.

In one embodiment of the present disclosure, as shown in FIG. 2A to FIG. 4, the operation process of the clamping device 100 may include the following steps: First, a robotic arm (not shown) is used to hold and move the syringe 200 to the clamping device 100. Next, the clamping driver 19 drives the first fixing member 11 and the second fixing member 12. The first fixing member 11 and the second fixing member 12 approach to each other, so that the first contact member 112 and the second contact member 122 are each in contact with the barrel 21 of the syringe 200. Afterwards, the first base body 111 and the second base body 121 are continuously driven to push the first connecting member 15 and the second connecting member 16, respectively. Next, the first connecting member 15 and the second connecting member 16 are respectively rotated to make the first pressing member 13 and the second pressing member 14 contact with the handle 22 of the syringe 200. As a result, it is able to achieve the purpose of using the clamping device 100 to clamp the syringe 200. Then, a pulling force or a pushing force may be applied to the push rod handle 231 of the syringe 200 to control the extraction or injection of the sample.

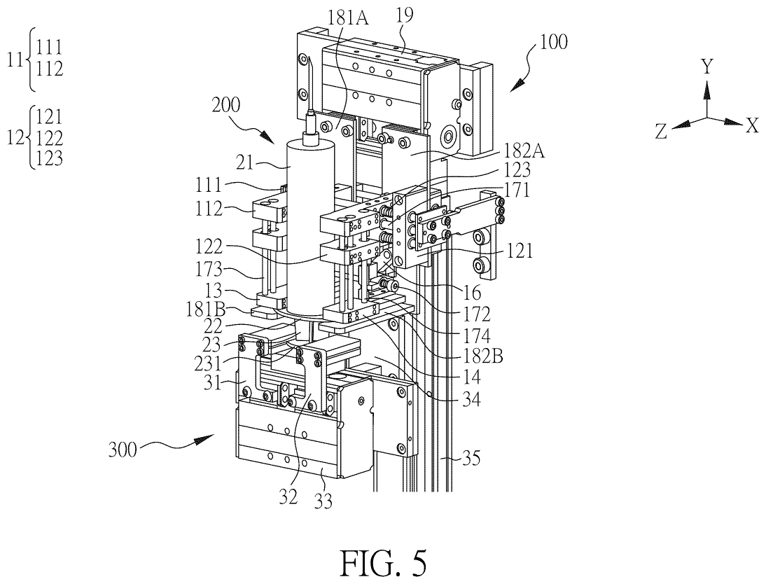

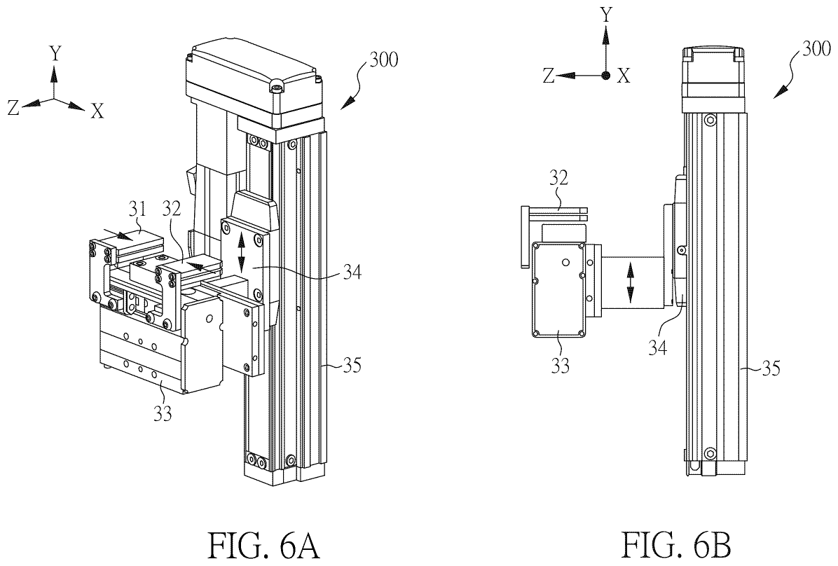

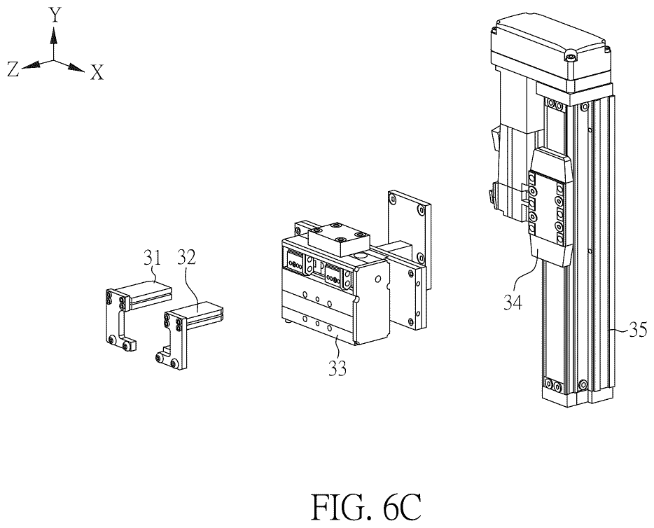

FIG. 5 is a three-dimensional schematic diagram of an electronic device according to an embodiment of the present disclosure. FIG. 6A is a three-dimensional schematic diagram of a push-pull device according to an embodiment of the present disclosure. FIG. 6B is a schematic side view of the push-pull device of FIG. 6A. FIG. 6C is an exploded view of the push-pull device of FIG. 6A.

In one embodiment of the present disclosure, as shown in FIG. 5, the electronic device may include: a clamping device 100 for clamping a syringe 200; and a push-pull device 300 adjacent to the clamping device 100. The clamping device 100 may be, for example, as shown in FIG. 2A to FIG. 2C, and the syringe 200 may be, for example, as shown in FIG. 3, which will not be described in detail herein.

The push-pull device 300 of the present disclosure will be described in detail below.

In one embodiment of the present disclosure, as shown in FIG. 5 to FIG. 6C, the push-pull device 300 may include: a first push rod clamp 31 and a second push rod clamp 32; and a push rod clamp driver 33, which is used to drive the first push rod clamp 31 and the second push rod clamp 32 so that the first push rod clamp 31 and the second push rod clamp 32 move closer to or farther away from each other along a first direction (that is, the X direction). In more detail, when the push rod clamp driver 33 drives the first push rod clamp 31 and the second push rod clamp 32, the first push rod clamp 31 approaches the second push rod clamp 32 in a first direction (for example, +X direction), and the second push rod clamp 32 approaches the first push rod clamp 31 in a direction opposite to the first direction (for example, −X direction), so that the first push rod clamp 31 and the second push rod clamp 32 approach each other and each contact the push rod 23 of the syringe 200. In detail, the first push rod clamp 31 and the second push rod clamp 32 may clamp and fix the push rod handle 231 of the syringe 200 when they approach each other.

In one embodiment of the present disclosure, as shown in FIG. 5 to FIG. 6C, the push-pull device 300 may further include a slide table 34 and a slide rail 35, wherein the slide table 34 is connected to the push rod clamp driver 33 and the slide 34 is connected in a sliding manner to the slide rail 35. The slide rail 35 extends in the longitudinal direction (that is, the Y direction) and allows the slide table 34 to move along the Y direction, that is, the slide rail 35 may extend along the second direction (for example, −Y direction) and/or along a direction opposite to the second direction (for example, +Y direction), and allows the slide table 34 to move along the second direction (for example, −Y direction) and/or along a direction opposite to the second direction (for example, +Y direction). The slide table 34 may drive the push rod clamp driver 33, the first push rod clamp 31 and the second push rod clamp 32 to move along the second direction (for example, −Y direction) and/or in a direction opposite to the second direction (for example, +Y direction). When the first push rod clamp 31 and the second push rod clamp 32 approach each other and each contact the push rod 23 of the syringe 200, the slide table 34 may move in the second direction (for example, −Y direction) to extract the sample, or move in in a direction opposite to the second direction (for example, +Y direction) to inject the sample. The second direction (for example, −Y direction) is perpendicular to the first direction (for example, +X direction). By moving the slide table 34 toward the second direction (for example, −Y direction) and/or in a direction opposite to the second direction (for example, +Y direction) to apply a pulling force or a pushing force to the push rod handle 231 of the syringe 200 to change the size of the space in the barrel 21, the syringe 200 may be controlled to extract or inject the sample.

In one embodiment of the present disclosure, as shown in FIG. 5 to FIG. 6C, the operation process of the electronic device may include the following steps: first, a robotic arm (not shown) is used to hold and move the syringe 200 to the clamping device 100 of the electronic device. Next, the clamping driver 19 drives the first fixing member 11 and the second fixing member 12. The first fixing member 11 and the second fixing member 12 approach to each other, so that the first contact member 112 and the second contact member 122 are each in contact with the barrel 21 of the syringe 200. Afterwards, the first base body 111 and the second base body 121 are continuously driven to push the first connecting member 15 (as shown in FIG. 2B) and the second connecting member 16, respectively. Next, the first connecting member 15 and the second connecting member 16 are respectively rotated to make the first pressing member 13 and the second pressing member 14 contact with the handle 22 of the syringe 200. As a result, it is able to achieve the automated operation of using the clamping device 100 of the electronic device to clamp the syringe 200. Then, the push rod clamp driver 33 drives the first push rod clamp 31 and the second push rod clamp 32 to approach each other, so that the first push rod clamp 31 and the second push rod clamp 32 are each in contact with the push rod 23 of the syringe 200; that is, the first push rod clamp 31 and the second push rod clamp 32 may clamp and fix the push rod handle 231 of the push rod 23 when they approach each other. Next, the slide table 34 is driven to apply a pulling force or a pushing force to the push rod handle 231 of the syringe 200, thereby controlling the syringe 200 to extract or inject a sample. As a result, the push-pull device 300 of the electronic device may realize the automated operation of extracting or injecting the sample.

In the present disclosure, a specially designed clamping device 100 and an electronic device including the clamping device 100 are used to make the clamping device 100 applicable to syringes 200 of various sizes, thereby achieving the effect of saving costs and/or simplifying operating steps.

The aforementioned specific embodiments should be interpreted as merely illustrative, and not limiting the rest of the present disclosure in any way.

1. A clamping device for clamping a syringe, comprising:
a first fixing member and a second fixing member;
a first pressing member and a second pressing member; and
a first connecting member and a second connecting member, wherein two ends of the first connecting member are pivotally connected to the first fixing member and the first pressing member, respectively, and two ends of the second connecting member are pivotally connected to the second fixing member and the second pressing member, respectively,
wherein the syringe includes a barrel and a handle and, when the first fixing member and the second fixing member approach to each other and contact the barrel of the syringe, the first pressing member and the second pressing member contact the handle of the syringe.

2. The clamping device as claimed in claim 1, wherein the first fixing member includes a first base body, a first contact member and a first elastic element, the first elastic element is arranged between the first base body and the first contact member, and the first fixing member contacts the barrel of the syringe through the first contact member.

3. The clamping device as claimed in claim 1, wherein, when the first fixing member and the second fixing member approach to each other, the first connecting member and the second connecting member are each pushed to rotate.

4. The clamping device as claimed in claim 3, wherein a rotation direction of the first connecting member is different from a rotation direction of the second connecting member.

5. The clamping device as claimed in claim 1, wherein the first fixing member approaches the second fixing member in a first direction, and the first pressing member contacts the handle of the syringe in a second direction, where the first direction is different from the second direction.

6. The clamping device as claimed in claim 5, wherein the first direction is perpendicular to the second direction.

7. The clamping device as claimed in claim 1, further comprising a clamping driver for driving the first fixing member and the second fixing member so that the first fixing member and the second fixing member approach to each other.

8. The clamping device as claimed in claim 2, wherein the second fixing member includes a second base body, a second contact member and a second elastic element, the second elastic element is arranged between the second base body and the second contact member, and the first base body, the second base body, the first contact member and the second contact member are each connected in a sliding manner to a main guide rod extending along a first direction.

9. The clamping device as claimed in claim 8, wherein the first elastic element and the second elastic element are each provided on the main guide rod.

10. The clamping device as claimed in claim 8, wherein the first contact member and the second contact member are each connected in a sliding manner to an auxiliary guide rod extending along the first direction.

11. The clamping device as claimed in claim 8, wherein a first guide rod extending along a second direction is provided on the first pressing member, a second guide rod extending along the second direction is provided on the second pressing member, the first pressing member is connected in a sliding manner to the first contact member through the first guide rod, and the second pressing member is connected in a sliding manner to the second contact member through the second guide rod, where the second direction is perpendicular to the first direction.

12. The clamping device as claimed in claim 8, further comprising a first back plate and a second back plate, wherein the first base body is fixed on the first back plate and the second base body is fixed on the second back plate.

13. The clamping device as claimed in claim 12, wherein the first back plate includes a first portion fixed thereon the first base body and extending along a second direction, and a second portion connected to the first portion and extending along a third direction, and the second back plate includes a third portion fixed thereon the second base body and extending along the second direction, and a fourth portion connected to the third portion and extending along the third direction, where the first direction is perpendicular to the second direction, the first direction is perpendicular to the third direction, and the second direction is perpendicular to the third direction.

14. An electronic device, comprising:
a clamping device for clamping a syringe, including:
a first fixing member and a second fixing member;
a first pressing member and a second pressing member; and
a first connecting member and a second connecting member, wherein two ends of the first connecting member are pivotally connected to the first fixing member and the first pressing member, respectively, and two ends of the second connecting member are pivotally connected to the second fixing member and the second pressing member, respectively; and
a push-pull device adjacent to the clamping device, including:
a first push rod clamp and a second push rod clamp; and
a push rod clamp driver for driving the first push rod clamp and the second push rod clamp,
wherein the syringe includes a barrel, a handle and a push rod and, when the first fixing member and the second fixing member approach to each other and contact the barrel of the syringe, the first pressing member and the second pressing member contact the handle of the syringe, and the first push rod clamp and the second push rod clamp approach each other and contact the push rod of the syringe.

15. The electronic device as claimed in claim 14, wherein the first fixing member includes a first base body, a first contact member and a first elastic element, the first elastic element is arranged between the first base body and the first contact member, and the first fixing member contacts the barrel of the syringe through the first contact member.

16. The electronic device as claimed in claim 14, wherein, when the first fixing member and the second fixing member approach to each other, the first connecting member and the second connecting member are each pushed to rotate, and a rotation direction of the first connecting member is different from a rotation direction of the second connecting member.

17. The electronic device as claimed in claim 14, wherein the first fixing member approaches to the second fixing member in a first direction, and the first pressing member contacts the handle of the syringe in a second direction, where the first direction is perpendicular to the second direction.

18. The electronic device as claimed in claim 14, wherein the clamping device further includes a clamping driver for driving the first fixing member and the second fixing member so that the first fixing member and the second fixing member approach to each other.

19. The electronic device as claimed in claim 14, wherein the push-pull device further includes a slide table connected to the push rod clamp driver.

20. The electronic device as claimed in claim 19, wherein the first push rod clamp approaches the second push rod clamp in a first direction, and the slide table moves in a second direction, where the second direction is perpendicular to the first direction.
